(19) United States
(12) Patent Application Publication
(10) Pub. No.: US 2012/0222200 A1
(43) Pub. Date: Sep. 6, 2012

(54) Protective Eyewear Device With Lateral Eye Access and Quick Release Mechanism for Interchanging Lenses

(76) Inventor: Arman Hedayat, Windermere, FL (US)

(21) Appl. No.: 13/392,715

(22) PCT Filed: Aug. 25, 2010
(86) PCT No.: PCT/US10/46597

§ 371 Date: May 14, 2012

(30) Foreign Application Priority Data
Aug. 25, 2009 (US) 12546718
Jul. 29, 2010 (US) 12845907

(51) Int. Cl. A61F 9/02

(52) U.S. Cl. 2/431

(57) ABSTRACT

In one embodiment, the present invention provides an eyewear device for wearing on the face of a user. The device includes a frame body with an interior portion, a front side, a rear side, a top portion and a bottom portion. The frame body also includes a first lateral appendage, a second lateral appendage, a first top lateral hingepoint and a first bottom lateral hingepoint, a second top lateral hingepoint and a second bottom lateral hingepoint. The frame body additionally includes a first distal side and a second distal side. The frame contacts the face of the user on the rear side, except at the first and second distal sides. When the hingepoints of the frame body are engaged, the first and second lateral appendages are rotatable toward said front side of the frame body.

### CROSS REFERENCE TO RELATED APPLICATIONS

This application claims priority to U.S. Ser. No. 12/845,907 filed Jul. 29, 2010, and U.S. Ser. No. 12/546,718, filed Aug. 25, 2009, which are incorporated herein by reference in their entirety.

### BACKGROUND OF THE INVENTION

Many different types of eyewear devices are used to protect the eye from foreign matter, while providing a view of the surroundings to the user. Such devices are often used during sports, both for water and non-water sporting activities.

It is often necessary for a user to be able to quickly change the lenses in these types of eyewear devices. Additionally, these eyewear devices often become foggy preventing the user from seeing his surroundings effectively. It is important to prevent fogging of the lenses of eyewear devices as well as provide a means to allow a user to access the eye-area during use of the eyewear device without completely removing the device from use. Restricted access to the eye while the eyewear device is in use is often necessary in order for a user to quickly swipe the eye area when small particulate matter or fluids may encounter the eye during use of the device.

### SUMMARY

According to one embodiment, the present invention provides a protective eyewear device for wearing on a user. The device includes a frame body with a front side, rear side, a bottom portion with a bottom side, and a top portion with a top side. The frame also includes a first and a second lateral end on each lateral side of the frame, and the frame is configured such that the frame contacts the face except at each lateral end. Additionally, the device includes a transparent lens secured onto the front side of the frame with a securing member to hold the frame onto the user's face such that said rear side of the frame is in contact with the face. The securing member attaches onto the frame at the first and second lateral ends of the frame to create a lateral opening on both first and second lateral ends of the frame in order to form a gap between the face of the user and the device, allowing the user access to the eye during use of the device.

In another embodiment, the present invention provides an eyewear device for wearing on the face of a user. The device includes a frame body with an interior portion, a front side, a rear side, a top portion and a bottom portion. In a more specific embodiment, the frame body includes a first and second appendage that may move from a closed position to an open position. The first appendage is proximate to a first lens and the second appendage is proximate to a second lens, whereby when in the open position, the lens may be removed from the frame.

In a more specific embodiment, the frame body includes a first lateral appendage, a second lateral appendage, a first top lateral hingepoint and a first bottom lateral hingepoint, a second top lateral hingepoint and a second bottom lateral hingepoint. The frame body additionally includes a first distal side and a second distal side. The frame contacts the face of the user on the rear side, except at the first and second distal sides. When the hingepoints of the frame body are engaged, the first and second lateral appendages are rotatable toward said front side of the frame body.

Additionally, in another embodiment, the device includes a right lens structure and a left lens structure, both which are slideably held in a groove in the bottom portion of the frame body. The first and second lateral appendages can be rotated about the hingepoints to release the lens structures.

A gap is formed between the face of the user and the eyewear device at the first and second distal sides when the eyewear device is in use. The gap allows a user to access the eye in order to remove debris from the eye or wipe the eye while the eyewear device is in use.

In another embodiment, the present invention provides an eyewear lens removal system. The system includes a frame body, made up of a top side, a bottom side, a rear side and a front side. The frame body also includes a first distal side which contains a first lateral appendage, and a second distal side which contains a second lateral appendage, wherein the first and second lateral appendages associate with the frame body at hingepoints. The system also includes a right lens structure and a left lens structure. The hingepoints of the system may be engaged by rotating the first and second lateral appendages in a front direction to release the right and left lens structures. This provides a quick release mechanism, allowing a user to easily remove and replace the lenses in the eyewear device.

In a further embodiment, the invention provides a method for the replacement of lenses on an eyewear device. The method includes the following steps: engaging the hingepoints by rotating the right and left lateral appendages about the hinge in a front direction; removing the lenses from the frame body by sliding them away from the center portion of the frame body. The next step involves inserting two new lenses into the groove in the bottom portion of the frame body, and slide the lenses toward the central portion of the frame body, and finally disengaging the hingepoints by rotating the left and right lateral appendages in a rear direction to secure the new lenses in place. The left and right lateral appendages may snap when they are rotated back into place once the lenses are secured.

### BRIEF DESCRIPTION OF THE DRAWINGS

A more particular description of the invention briefly described above will be rendered by reference to specific embodiments thereof that are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 depicts a front view of an exemplary embodiment of a protective eyewear device

FIG. 2 represents a cross-section view of the device shown in FIG. 1 along the plane 9-9.

FIG. 3 shows a side view of the embodiment shown in FIG. 1 illustrating the gap formed by the lateral opening on the side of the protective eyewear device.

FIG. 4 depicts a top view of an exemplary embodiment of a protective eyewear device.

FIG. 5 depicts a side rear view of the an embodiment of a protective eyewear device.

FIG. 6 shows an embodiment of a protective eyewear device that has a securing member that includes two separate bands.

FIG. 7 shows a front view of an alternative embodiment of a protective eyewear device.

FIG. 8 shows a top view of the embodiment shown in FIG. 7.

FIG. 9 depicts a perspective view of an exemplary embodiment of an eyewear device.

FIG. 10 depicts a rear side view of the frame body of an exemplary embodiment of an eyewear device.

FIG. 11 shows a rear side view of an exemplary embodiment of an eyewear device with the hingepoints engaged to allow removal and/or replacement of the lens structures.

FIG. 12 represents a rear view of an exemplary embodiment of the frame body.

FIG. 13 depicts a perspective view of an exemplary embodiment of both right and left lens structures.

FIG. 14 represents a cross-section view of the device shown in FIG. 4 along the plane x-x.

FIG. 15 represents a side perspective view of an embodiment in use.

### DETAILED DESCRIPTION OF THE DRAWINGS

A common problem associated with most eyewear devices, particularly those that are worn close to the face such as sport goggles, is that they tend to fog while in use. There are many different types of anti-fogging mechanisms manufactured for use in goggles in order to counteract this problem. However, they often malfunction, and as with all mechanisms, they require maintenance. Also, the inventor has realized an additional problem with these goggles, particularly when they used in water sports, water tends to accumulate inside the goggle between the user's face and the lens. This obstructs the ability of the user to see clearly, as well as resulting in discomfort for the user.

An additional concern associated with the use of goggles, is that often debris or water gets trapped inside the goggle and cannot be removed without removing the entire goggle from the face of the user. The ability of the user to access the protected eye area if necessary to swipe the water or debris away without removal of the entire goggle is ideal.

In accordance with another aspect of the present invention, there is provided a device configured to protect the eyes of a user from debris and other foreign bodies, while also providing eye-access to the user while in use. The inventor has recognized a need for such a device, particularly for use in sports such as wakeboarding, kite boarding, and other water-sport activities where a completely sealed goggle structure is not necessary, but eye protection would be advantageous. In such water-sport activities, protection to the eye from both wind and foreign particles is ideal, and quick and easy restricted access to the eye is beneficial. The inventor has also recognized a need for such a device during non-water sport activities such as racquetball or tennis. The device would be useful in order to protect the eye, but also allow a user quick access to the eye area through a restricted opening in order to wipe away any sweat or other particulate matter that may encounter the eye.

In one embodiment, the invention pertains to a protective eyewear device for wearing on a face of a user, which allows access to the eye without having to remove goggles from over the eyes. The embodiment allows eye access at each lateral end of the protective eyewear device. In a specific embodiment, the device includes a frame body that has a front side, a rear side, a bottom side, a top side, and a center portion. The frame has a first lateral end and a second lateral end and is configured such that the frame contacts the face except at the first and second lateral ends. The frame includes includes a transparent lens that is associated with the frame such that it shields the eyes of the user from a front side direction. The frame also includes a securing member associated therewith that holds the device onto the face such that said rear side of said frame is in contact with the face. The securing member attaches onto the frame at said first and second lateral ends of said frame in such a fashion as to create a lateral opening on both said first and second lateral ends of the frame. The device is configured to meet the face of the user such that the frame forms a gap between the face of the user and the device to allow access to the eye during use of the device. Moreover, the securing member is associated with the frame and is configured such that it does not obstruct this gap.

In a more specific embodiment, the back side of said frame is covered by a foam backing layer which is thicker at each said first and second lateral ends of said frame and gradually becomes thinner at the center of said frame. In another embodiment, the lens is removable and is attached onto the frame to form a space between the lens and the frame; and the lens is mounted onto said frame with flushmount or flat head stainless steel screws. In yet another embodiment, the securing member is comprised of a top band and a bottom band, wherein the top band connects to the first and second lateral ends proximate to or on the top side of the frame, and the bottom band connects to the first and second lateral ends proximate to or on the bottom side of said frame to secure the device to the face of the user. In an alternative embodiment, the securing member is comprised of a band body with diverging band ends on each side of the band body, wherein one end of the band attaches to the first lateral end of the frame and the other end of the band attaches to the second lateral end of the frame to secure the device to the face of the user.

In a more specific embodiment, the gap between the frame and the face of the user described above is between 1-6 centimeters depending on the facial structure of the user. In an even more specific embodiment, the gap is between 2-4 centimeters depending on the facial structure of the user.

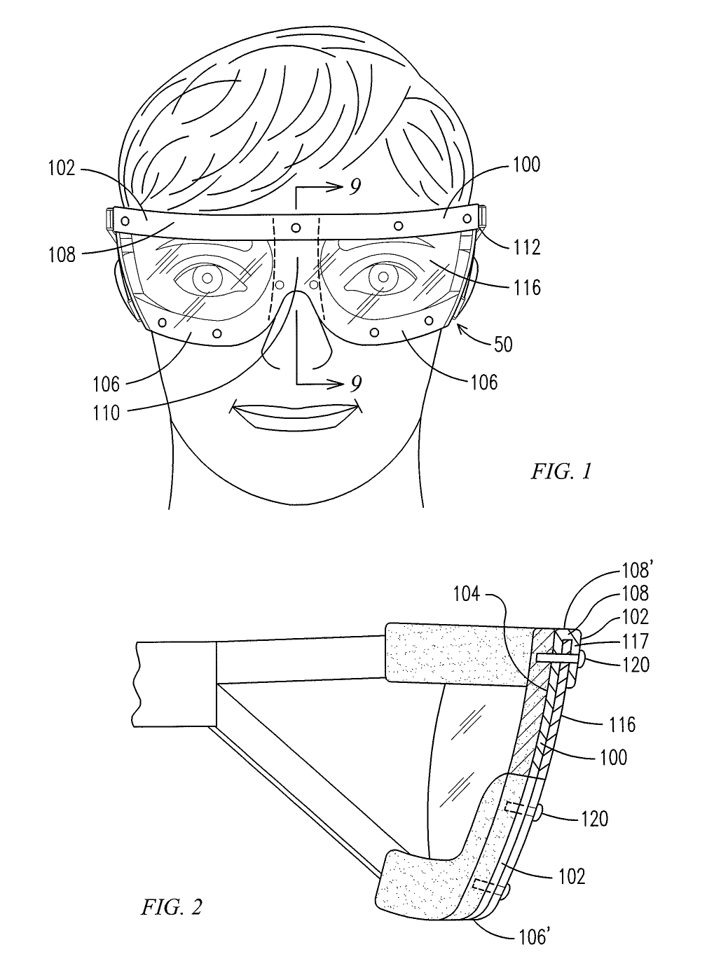

Turning to the drawings, FIG. 1 shows a front view of a protective eyewear device 50. The device includes a frame body 100 that has a front side 102 and a center portion 110 (dashed lines). Associated with the frame body 100 is a transparent lens 116. Also shown on this view is a first lateral end 112 of the frame body 100. FIG. 2 shows cross-sectional view on plane 9-9 from FIG. 1. As shown in this view, the frame body 100 has rear side 104, bottom portion 106 having a bottom side 106′, and top portion 108 having a top side 108′. This view shows that the lens 116 fits into a groove 117 defined in the top portion 108 of the frame 100. The lens 116 rests against the front side 102 of the frame at the bottom portion 106. The lens 116 is attached to the frame 100 for example by screws 120 (or other suitable fastener such as but not limited to, rivets, pins, staples, dowels etc. or alternatively via an adhesive thereby alleviating the need for fasteners, though fasteners and adhesives can be used together).

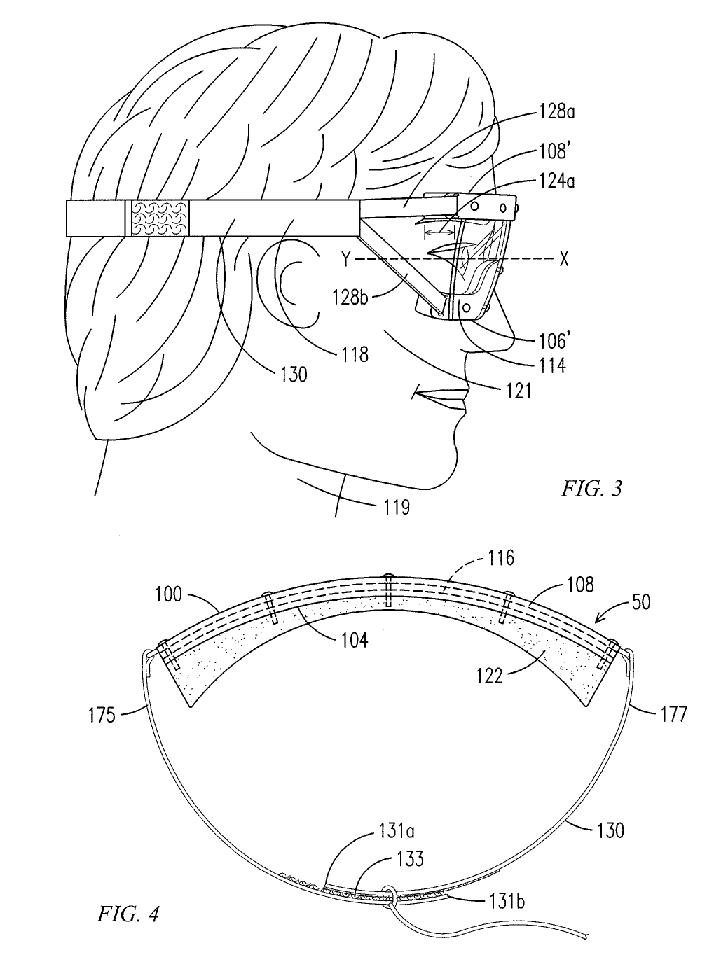

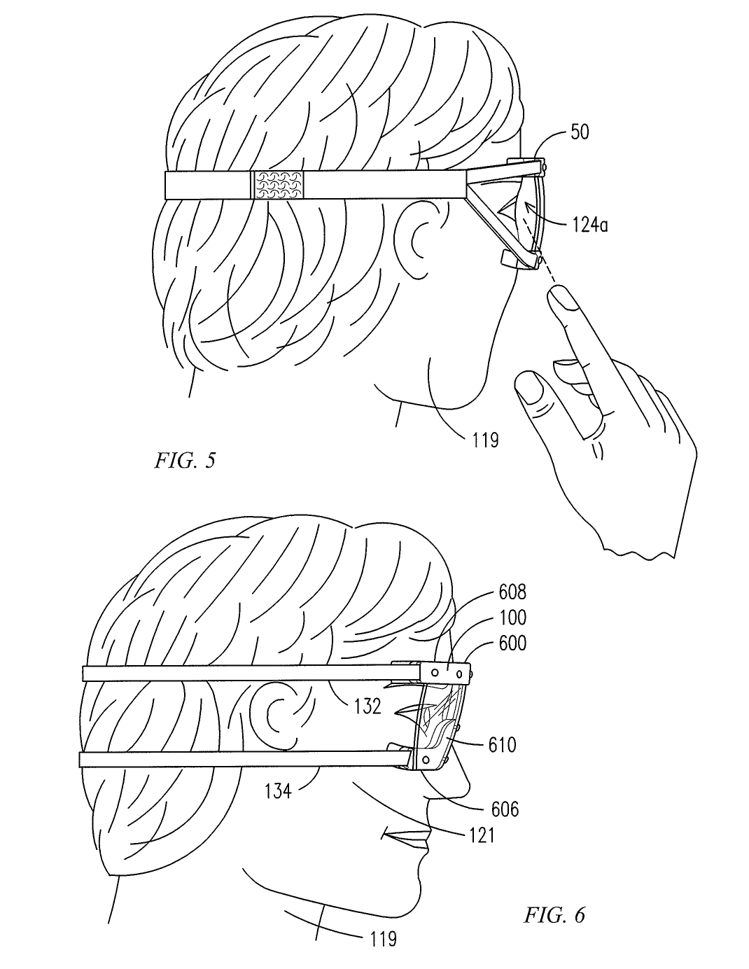

FIG. 3 shows a side view showing a second lateral end 114 of the protective eyewear device 50. Attached to the frame body 100 is a securing member 118. The securing member 118 has a band body 130 with a first divergent end 128a attaching proximate to the top portion 108 and a second divergent end 128b attaching proximate to the bottom portion 106. The first and second divergent ends 128a,b are provided so as to not obstruct the lateral gap 124a formed by the protective eyewear device 50 and face 121 of the user. FIG. 5 shows a side rear view which better illustrates the gap 124a. The gap 124a gives the user 119 access to wipe water from the eyes. However, the protective eyewear device 50 is sealed upon the user's face 121 at the top and bottom sides. Thus, the protective eyewear protects the eyes from turbulent splashing into the eyes, while preventing fogging experienced by convention goggles and enables the user 119 to clean their eyes without having to remove the protective eyewear device 50.

FIG. 4 shows a top view of the device embodiment 50. As can be seen in this view, the lens 116 dashed lines is shown fitting into the top portion 108 of the frame body 100 with screws 120. Those skilled in the art will appreciate that numerous other fasteners may be used when equipped with the teachings herein. Provided on the rear side 104 of the frame body 100 is a cushion pad layer 122. The band body 130 of the securing member 118 comprises opposing ends 175, 177. More specifically, as shown the band body 130 comprises a first band component 131a and a second band component 131b. The band components 131a,b are secured together by a hook and loop material 133.

In between the lens 116 and frame body 100 is a space 126 to allow air to flow in and out between the device 50 and the user (not shown). This air flow prevents fogging of the lens 116. The gap is formed by spacers 121 situated between the lens 116 and the frame body 100 at the screws 120. The frame body 100 has a securing member 118 attached at a first lateral end 112 and at the second lateral end 114 via a ring 138a and 138b, respectively. Associated with the securing member 118 is a neck strap 136 which may attach somewhere else on the user or user clothing as a leash to avoid loss of the device 50. Also shown in this view is the cushion pad layer 122. This view reveals that the cushion pad layer 122 tapers down from the first and second lateral ends 112, 114 to the center portion 110 of the frame body 100. At the first and second lateral ends 112, 114, a lateral gap 124a and 124b is defined which enables access to the eye while the device 50 is situated on the user's face.

As used herein, the term “proximate” when used with respect to the bottom side of the structure, (proximate to the bottom side) means somewhere between the centerline x-x′ axis (see FIG. 3) and the bottom side of a lateral end of the structure. “Proximate” when used with respect to the top side of the structure, (proximate to the top side) means somewhere between the centerline x-x′ axis (see FIG. 3) and the top side of a lateral end of the structure.

FIG. 6 shows a side view of an alternative embodiment of a protective eyewear device 600. According to this embodiment, the means for securing the device 600 to the face 121 of a user 119 includes a top band 132 and a bottom band 134. This is in place of the divergent band ends discussed in relation to FIG. 1. Important to this invention is that the top band 132 is attached to the frame 610 proximate to the top side 608 and the bottom band 134 is attached to the frame 610 proximate to the bottom side 606. This arrangement prevents the bands 132, 134 from obstructing the lateral gap 124a formed between the device 600 and the user's face 121.

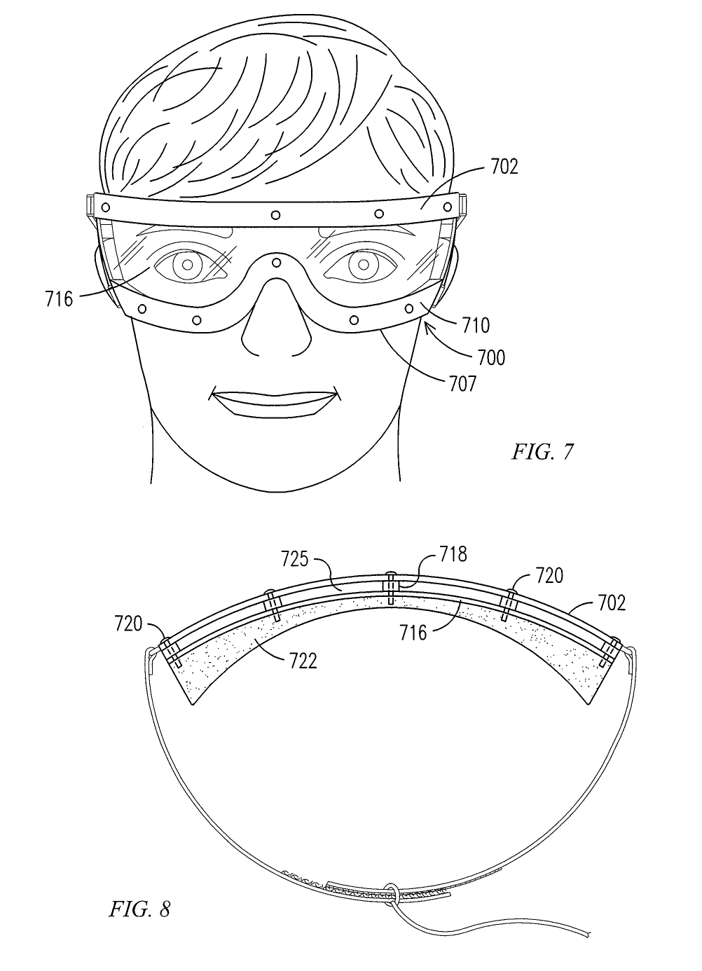

FIG. 7 shows a front view and FIG. 8 shows a top view of an alternative eyewear protective device embodiment 700. This embodiment 700 comprises a frame 710 that has a top portion 702 and a bottom portion 704 but lacks a center portion. The embodiment 700 includes a lens 716 that is attached to both top and bottom frame portions 702, 704. The top portion 702 is attached to the lens 716 by screws 720. Spacers 718 are provided between the top portion 702 which creates a ventilation space 725 between the lens 716 and the top portion 702. Note that the spacers 718 are optional and maybe included between the bottom portion (not shown) and the lens 716 as well. Associated with the back side of the lens 716 is a cushion pad layer 722.

A further concern with goggles and other eyewear devices is the inability to easily remove and replace the lenses when necessary. Typically, the removal of lenses from eyewear devices requires certain assembling or disassembling tools such as miniature screw-drivers or other small difficult-to-find contraptions. These type of tools are not always readily available to a user when a lens needs to be exchanged. Removing or replacing lenses in eyewear is often inconvenient and time consuming.

In one embodiment, the invention pertains to an eyewear device for wearing on the face of a user, which allows the user to access the eye without removing the device. In a particular embodiment the device includes a frame body with an interior portion, a front side, a rear side, a top portion and a bottom portion. The frame also includes a first lateral appendage and a second lateral appendage, a first top lateral hingepoint and a first bottom lateral hingepoint, a second top lateral hingepoint and a second bottom lateral hingepoint. The frame also includes a first distal side and a second distal side, and is configured such that the frame contacts the face of the user on said rear side, except at the first and second distal sides of the frame body. Moreover, when the hingepoints of the frame are engaged, the first and second lateral appendages are rotatable toward the front side. Additionally, the device also includes a right lens structure and a left lens structure, which are slideably held in a groove in the bottom portion of the frame body. The first and second lateral appendages can be rotated about the hingepoints providing release of the lens structures. The device provides a gap, formed between the face of the user and the eyewear device at the first and second distal sides of the frame body when the eyewear device is in use. In an alternative embodiment, the frame body is flexible.

In a more particular embodiment, the hingepoints rotate frontally at least 20 degrees, and more preferably 30-90 degrees. In yet another embodiment, the right and left lens structures have a distal and a proximal side. The distal side is greater in length than the proximal side of each lens structure. In still another embodiment, the interior portion of the frame body contains a groove pocket at the top portion and the bottom portion of the frame body. The shape and structure of the lens allows it to be easily removed from or inserted into the frame body by simply sliding it one way or the other within the groove pocket in the interior portion of the frame body.

In a more specific embodiment, the gap is from about 1 centimeter to about 5 centimeters, or any 0.2 centimeters in between. In another embodiment of the present invention, a flexible band attaches to the first and second lateral appendages to secure the eyewear device onto the face of the user.

In yet another embodiment, an eyewear lens removal system is provided. The system includes a frame body with a top side, a bottom side, a rear side and a front side. The frame also contains a first distal side with a first lateral appendage, and a second distal side with a second lateral appendage, the first and second lateral appendages associate with the frame body at hingepoints. The system also includes a right lens structure and a left lens structure. The hingepoints may be engaged by rotating the first and second lateral appendages in a front direction in order to release the right and left lens structures for replacement of the lenses. In a more specific embodiment, the right and left lens structures of the system are secured within the frame body in a groove pocket.

In another embodiment, the distance between the distal side of the frame body and the hingepoint is “y”. In a more particular embodiment, the distance “y” is between 3-5 millimeters in length. In an alternative embodiment, “y” extends from the distal side of the frame body to about 2 millimeters inside the distal side of the lens structure.

In another embodiment of the present invention, a method is provided for the replacement of lenses on an eyewear device. The method includes first engaging the hingepoints by rotating the right and left lateral appendages about the hinge in a front direction; removing the lenses from the frame body by sliding them away from the center portion of the frame body. The method also includes inserting two new lenses into the groove in the bottom portion of the frame body, and sliding the lenses toward the central portion of the frame body; and finally disengaging the hingepoints by rotating the left and right lateral appendages in a rear direction to secure the new lenses in place. Once the lenses are secured in place, the left and right lateral appendages may snap into position.

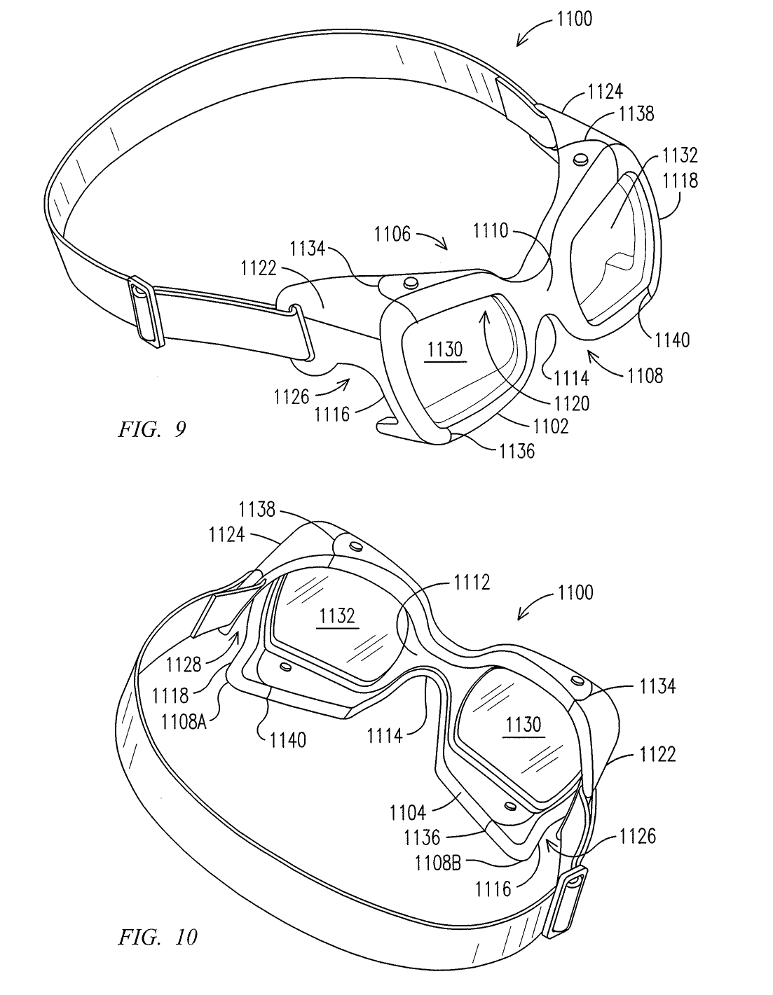

Turning now to the drawings, FIG. 9 shows a perspective view of an exemplary embodiment of an eyewear device 1100. The device includes a frame body 1102, with a top portion 1106, a bottom portion 1108, a first distal side 1116 and a second distal side 1118. The frame body also includes a front side 1110, and a center portion 1114, as well as an interior portion 1120. Associated with the frame is a first lateral appendage 1122 and a second lateral appendage 1124, wherein the intersection between the first lateral appendage 1122 and the first distal side of the frame 1116 forms a right lateral gap 1126, and the second lateral appendage 1124 intersects with the second distal side of the frame 1118 (behind the device in the picture) to form a left lateral gap.

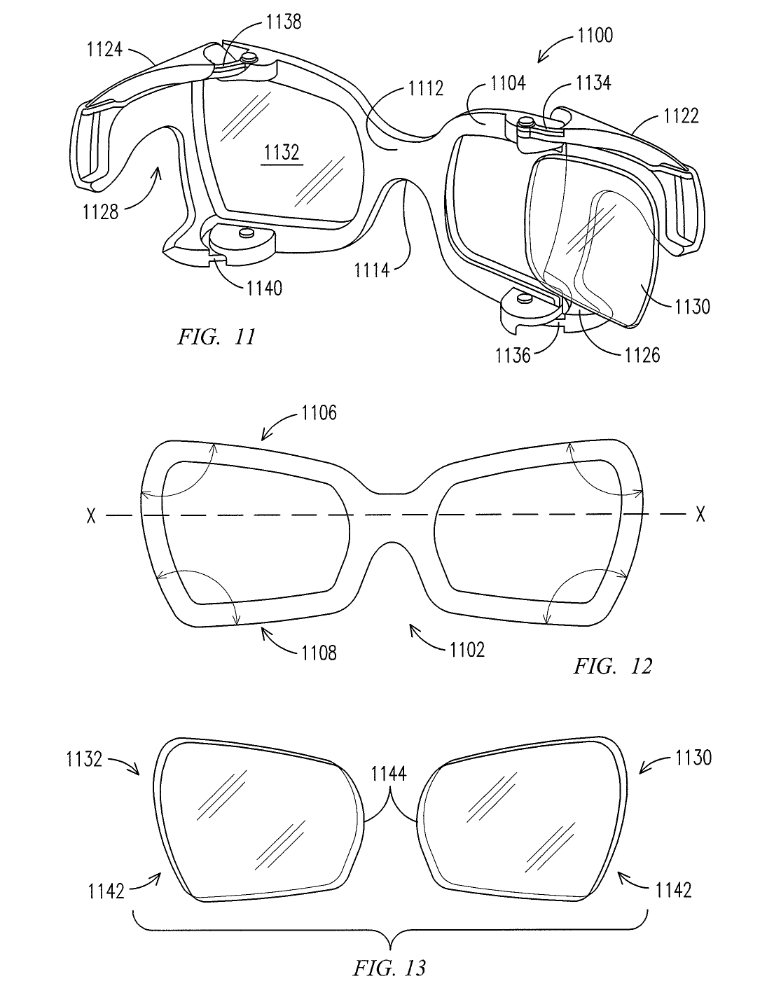

FIG. 10 depicts the rear side 1112 in a rear side view of the frame body 102 of an exemplary embodiment of an eyewear device 1100. The first top lateral hingepoint 1134 and first bottom lateral hingepoint 1136, as well as the second top lateral hingepoint 1138 and the second bottom lateral hingepoint 1140 are shown. All hingepoints in this figure are shown as disengaged. A right lens structure 1130 and a left lens structure 1132 are seen in the figure surrounded by the frame body 1102. Both the right lateral gap 1126 and the left lateral gap 1128 are in view in this figure. The right lateral gap 1126 is defined between the right lateral appendage 1122 and bottom portion 1108a and the left lateral gap 1128 is formed between second lateral appendage 1124 and bottom portion 1108b. In an optional embodiment, a cushion pad layer may be applied to the region 1104 layering the rear side 1112, so as to increase comfort and to provide contact between the face of a user and the frame body 1102 of the device 1100. FIG. 11 shows the rear side 1112, a rear side view of an exemplary embodiment of an eyewear device 1100, with the hingepoints 1134, 1136, engaged to allow removal and/or replacement of the lens structure 1130.

FIG. 12 represents a rear view of an exemplary embodiment of the frame body 102, showing the acute angles formed by the top portion 1106 and the bottom portion 108 of the frame with the first and second distal sides of the frame 1116, 1118. An x-x axis is also represented in FIG. 12.

FIG. 13 depicts a perspective view of an exemplary embodiment of both right and left lens structures, 1130 and 1132. The embodiment represents the distal sides of the lens structures 1142 are greater in length than the proximal sides of the lens structures 1144 in order to facilitate easier removal of the lenses 1130, 1132 from the frame body 1102.

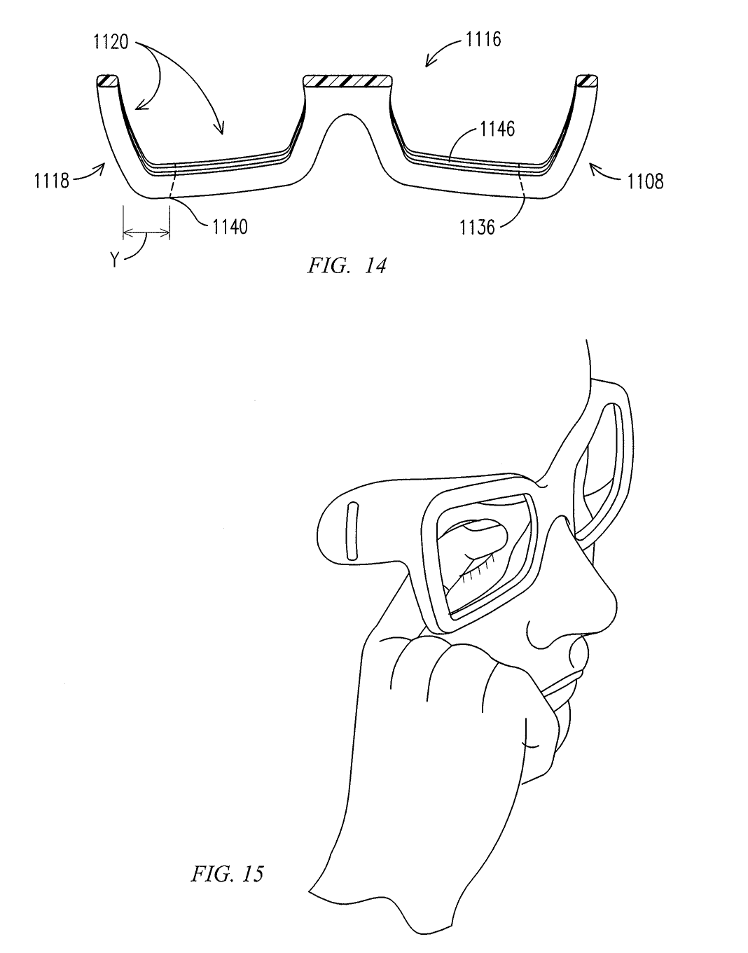

FIG. 14 represents a cross-section view of the device 1′00 shown in FIG. 12 along the plane x-x, with the groove pocket 1146 visible within the interior portion 1120 of the frame body 1102. The groove pocket 1146 assists in keeping the lens structures 1130, 1132 in place within the frame body 1120 of the eyewear device 1100.

In another embodiment, the invention pertains to an eyewear device that has the structural shape and configuration of that shown in FIG. 9, but which lacks hingepoints and a lens release mechanism.

In another embodiment, the eyewear device comprises a movable portion on the top portion or bottom portion of the frame so as to enable removal and replacement of lenses. For example, in one embodiment the frame includes a hinged piece at the top portion which can opened to remove the lens. The hinged piece can include a first hinged piece above one lens and a second hinged piece above the other lens. Alternatively, the bottom portion comprises a hinged piece for access to the lens, or two separate pieces below each lens. Alternatively, the movable portion is a piece or two pieces that removably snap onto the top portion or bottom portion.

FIG. 15 shows a eyewear device shown in a model to illustrate how the shape and configuration of the device facilitate access to the eyes for wiping and cleaning but which also provides protection to the eye.

The disclosures of the cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims

What is claimed is:

1. A protective eyewear device for wearing on a face of a user, said device comprising:
a frame body comprising a front side, a rear side, a bottom portion having a bottom side, a top portion having a top side, and a center portion; a first lateral end and a second lateral end on each lateral side of said frame, the frame being configured such that the frame contacts the face except at the first and second lateral ends,
a transparent lens secured to said frame,
a securing member for holding said frame onto the face such that said rear side of said frame is in contact with the face; said securing member attaches onto said frame at said first and second lateral ends of said frame to create a lateral opening on both said first and second lateral ends of said frame forming a gap between the face of the user and the device to allow access to the eye during use of the device.

2. A device as in claim 1 wherein said rear side of said frame comprises a cushion pad which is thicker at each said first and second lateral ends of said frame and gradually becomes thinner at the center of said frame, wherein the cushion pad of the frame contacts the face except at the first and second lateral ends.

3. A device as in claim 1 wherein said lens is removable and is attached onto said frame to form a space between said lens and said frame; and said lens is mounted onto said frame with flushmount or flat head stainless steel screws.

4. A device as in claim 1 wherein said securing member is comprised of a top band and a bottom band, wherein said top band connects to said first and second lateral ends proximate to or on the top side of said frame, and said bottom band connects to said first and second lateral ends proximate to or on the bottom side of said frame to secure the device to the face of the user.

5. A device as in claim 1 wherein said securing member is comprised of a band body with diverging band ends on each side of said band body, wherein each band end attaches to said first and second lateral ends of said frame to secure the device to the face of the user.

6. A device as in claim 1 wherein said gap is between 1-6 centimeters depending on the facial structure of the user.

7. A device as in claim 4 wherein said gap is between 2-4 centimeters depending on the facial structure of the user.

8. A protective eyewear device for wearing on a face of a user, said device comprising:
a frame body comprising a front side, a rear side, a bottom portion having a bottom side, a top portion having a top side, and a center portion; a first lateral end and a second lateral end on each lateral side of said frame;
a transparent lens secured onto the frame, and wherein said lens rests in a groove defined within the top portion and rests on the front side of the frame at the bottom portion;
a cushion pad associated with the rear side of the frame, wherein the frame and cushion pad are configured such that the cushion pad contacts the face except at the first and second lateral ends; and
a securing member for holding said frame onto the face such that said rear side of said frame is in contact with the face; said securing member comprising a band body having opposing ends where the opposing ends each have a first diverging end that associates with said frame proximate to said top side and a second diverging end that associates with said frame proximate to said bottom side so as to create a lateral opening on both said first and second lateral ends of said frame, wherein the frame, cushion pad and securing member create a gap between the face of the user and the device to allow access to the eye during use of the device.

9. A device as in claim 8, wherein said gap is from about 1 centimeter to about 5 centimeters, or any 0.2 centimeter increments in between.

10. A device as in claim 8, wherein said band body comprises a first band body portion that associates with said first lateral end and a second band body portion that associates with the second lateral end, wherein said first and second band body portions engage via a hook and loop material.

11. A device as in claim 8, wherein said lens is secured to the frame by a plurality of fasteners.

12. A device as in claim 8, wherein said lens is secured to the frame via an adhesive.

13. An eyewear device for wearing on the face of a user, said device comprising:
a frame body comprising an interior portion, a front side, a rear side, a top portion and a bottom portion, a first lateral appendage and a second lateral appendage, a first top lateral hingepoint and a first bottom lateral hingepoint, a second top lateral hingepoint and a second bottom lateral hingepoint, a first distal side and a second distal side, wherein the frame contacts the face of the user on said rear side, except at the first and second distal sides; wherein when said hingepoints are engaged, said first and second lateral appendages are rotatable toward said front side;
a right lens structure and a left lens structure slideably held in a groove in said bottom portion of said frame body, whereby said first and second lateral appendages can be rotated about said hingepoints to release said lens structures; and
a gap defined between said first and second lateral appendage and said bottom portion such that an access is provided between the face of the user and the eyewear device when worn.

14. A device as in claim 13, wherein said frame body is flexible.

15. A device as in claim 13, wherein said hingepoints rotate frontally at least 70 degrees.

16. A device as in claim 13, wherein the top portion and the distal sides of the frame body join at acute angles.

17. A device as in claim 13, wherein the bottom portion and the distal sides of the frame body join at acute angles.

18. (canceled)

19. A device as in claim 13, wherein the interior portion of the frame body contains a groove pocket at the top portion and the bottom portion of the frame body.

20. A device as in claim 13, wherein said gap is from about 1 centimeter to about 5 centimeters, or any 0.2 centimeters in between.

21. A device as in claim 1, wherein a flexible band attaches to the first and second lateral appendages to secure the eyewear device onto the face of the user.

22-29. (canceled)
